(19) United States
(12) Patent Application Publication
(10) Pub. No.: US 2023/0201273 A1
(43) Pub. Date: Jun. 29, 2023

(54) NOVEL DEINOCOCCUS RADIODURANS STRAIN, EXOPOLYSACCHARIDE DERIVED THEREFROM, AND COMPOSITION COMPRISING THE SAME

(71) Applicant: KOREA ATOMIC ENERGY RESEARCH INSTITUTE, Daejeon (KR)

(72) Inventors: Ho-Seong SEO, Jeongeup-si, Jeollabuk-do (KR); Hae-Ran PARK, Jeonju-si, Jeollabuk-do (KR); Sang-Yong LIM, Daejeon (KR); Jong-Hyun JUNG, Seoul (KR); Chan-Yu BAEK, Changwon-si, Gyeongsangnam-do (KR); Ji-Hee LEE, Damyang-gun, Jeollanam-do (KR); Dong-Ho KIM, Jeongeup-si, Jeollabuk-do (KR); Min-Kyu KIM, Jeongeup-si, Jeollabuk-do (KR); Eui-Baek BYUN, Daejeon (KR); Ki-Bum AHN, Jeonju-si, Jeollabuk-do (KR); Ha-Yeon SONG, Seoul (KR); Hyun-Jung JI, Jeongeup-si, Jeollabuk-do (KR)

(21) Appl. No.: 17/916,005

(22) PCT Filed: Mar. 29, 2021
(86) PCT No.: PCT/KR2021/003811

§ 371 Date: Sep. 29, 2022

(30) Foreign Application Priority Data
Mar. 30, 2020 (KR) 10-2020-0038472
Mar. 26, 2021 (KR) 10-2021-0039384

(51) Int. Cl. A61K 35/74; A61Q 19/08; A23L 33/135; A61K 8/99

(52) U.S. Cl. CPC A61K 35/74; A61Q 19/08; A23L 33/135; A61K 8/99

(57) ABSTRACT

A novel Deinococcus radiodurans strain, an exopolysaccharide derived therefrom and a composition comprising the same are provided. In detail, a Deinococcus radiodurans BRD125 strain characterized in being deposited with accession number KCTC 13955BP, an exopolysaccharide derived therefrom and a composition comprising the same, and a method of extracting a Deinococcus radiodurans-derived exopolysaccharide are provided.

### TECHNICAL FIELD

The present disclosure relates to a novel Deinococcus radiodurans strain, an exopolysaccharide derived therefrom, and a composition comprising the same, and more particularly, to a novel Deinococcus radiodurans strain producing significantly increased amount of exopolysaccharides providing excellent antioxidant performance, and protective effects for cell and living body against UV/X-rays/gamma rays, and a technology related to exopolysaccharides thereof.

### BACKGROUND ART

Deinococcus radiodurans is a Gram-positive bacterium as almost the only microorganism that may survive not only under desiccation conditions, but also under ultraviolet and ionizing radiation conditions, and it is reported that growth thereof is possible under such conditions because the DNA repair system of the strain itself is superior to cells of other organisms along with specific hydrophobic substances and carotenoids in the cell membrane.

Korean Patent Registration No. KR10-1776586 B1 or the like discloses that an exopolysaccharide derived from Deinococcus radiodurans, which may survive in extreme environments as described above, exhibits effects such as skin aging inhibition, skin recovery and the like.

On the other hand, repeated and prolonged exposure to irritants from the environment denatures skin proteins, degrades the lipid lamella layer, removes protective intercellular lipids, makes to lose natural moisturizing factors, and reduces intercellular adhesion. For example, UV exposure causes damage to the epidermis and dermis, and chronic exposure to UV causes a modification of the dermal biomechanical properties that cause wrinkles. An exopolysaccharide derived from Deinococcus radiodurans may be applied in various types of compositions, such as a cosmetic composition having anti-aging and skin recovery effects.

However, in order to commercially apply the exopolysaccharide derived from Deinococcus radiodurans in this manner, a sufficient yield of exopolysaccharide production should be obtained, and further, it is required that exopolysaccharides can exert improved efficacy.

Accordingly, the present inventors studied a new strain of Deinococcus radiodurans, and particularly confirmed Deinococcus radiodurans, which produces exopolysaccharides having excellent antioxidant efficacy and remarkably improved yield, and the produced exopolysaccharide provided excellent antioxidant performance and cellular and bioprotective effects against UV/X-rays/gamma rays, thereby completing the present disclosure.

### DISCLOSURE

### Technical Problem

An aspect of the present disclosure is to provide a novel Deinococcus radiodurans strain producing exopolysaccharides having excellent antioxidant efficacy in a significantly improved yield.

Another aspect of the present disclosure is to provide an exopolysaccharide having excellent antioxidant and radioprotective efficacy.

Another aspect of the present disclosure is to provide a composition comprising an exopolysaccharide having excellent antioxidant and radioprotective efficacy.

Another aspect of the present disclosure is to provide a method of extracting exopolysaccharides having excellent antioxidant and radioprotective efficacy, from Deinococcus radiodurans.

### Technical Solution

According to an aspect of the present disclosure, a Deinococcus radiodurans BRD125 strain characterized in being deposited with accession number KCTC 13955BP is provided.

According to an aspect of the present disclosure, an exopolysaccharide derived from a Deinococcus radiodurans BRD125 strain deposited with accession number KCTC 13955BP is provided.

According to another aspect of the present disclosure, a cosmetic composition, a pharmaceutical composition for inhibiting skin aging, a pharmaceutical composition for skin recovery and an antioxidant food, including an exopolysaccharide derived from a Deinococcus radiodurans BRD125 strain, are provided.

According to another aspect of the present disclosure, a method of extracting an exopolysaccharide derived from Deinococcus radiodurans includes obtaining a culture medium by culturing a Deinococcus radiodurans BRD125 strain; precipitating the exopolysaccharide in the culture medium, using an ethanol aqueous solution having an ethanol concentration of 50% or more; an impurity removal operation of removing proteins, lipids, and nucleic acids as precipitates from a precipitate of the precipitating; and purifying a supernatant obtained in the impurity removal operation.

### Advantageous Effects

In the case of the novel Deinococcus radiodurans strain provided according to an exemplary embodiment of the present disclosure, the production of exopolysaccharides may be significantly increased, compared to the related art strain, and further, an exopolysaccharide derived from the novel Deinococcus radiodurans strain according to an exemplary embodiment has excellent antioxidant effects and excellent cellular and bioprotective effects against UV/X rays/gamma rays, thereby being expected to be widely applied in various fields industrially.

### DESCRIPTION OF DRAWINGS

FIG. 1 illustrates a satellite image of a soil collection site for separating radiation-resistant microorganisms.

FIG. 2 schematically illustrates a radiation-resistant microorganism separation process according to an exemplary embodiment of the present disclosure.

FIG. 3 illustrates the amount of DeinoPol produced per Deinococcus radiodurans strain.

FIG. 4 schematically illustrates the DeinoPol production process according to an exemplary embodiment of the present disclosure.

FIG. 5 illustrates the results of comparing the antioxidant effects of DeinoPol BRD125 and DeinoPol R1.

FIG. 6 illustrates the results of comparison of protection effects of DeinoPol BRD125 and DeinoPol R1 against UV.

FIGS. 7A and 7B illustrate the results of radiation (X-ray)-induced reactive oxygen species scavenging ability (FIG. 7A) and cell viability against radiation (FIG. 7B) in CHO cells by DeinoPol BRD125.

FIGS. 8A to 8C illustrate the inhibitory ability of DeinoPol of the novel strain BRD125 against cell death by radiation in bone marrow cells and immune cells in the spleen, and in more detail, illustrates changes in the cell cycle ratio by radiation (X-ray) irradiation in bone marrow cells and immune cells in the spleen (FIG. 8A, 8B) and the result of an increase in the expression of BCL-2, a cell death inhibitory factor, by DeinoPol (FIG. 8C).

FIGS. 9A to 9C illustrate the inhibitory ability of DeinoPol of the novel strain BRD125 on the death of bone marrow cells (FIG. 9A) and splenic immune cells (FIG. 9B) of irradiated mice (gamma rays), and the inhibitory effect of DeinoPol on the DNA fragmentation of bone marrow cells of irradiated mice (FIG. 9C).

FIG. 10 illustrates the results of increasing the formation of endogenous spleen colonies by DeinoPol of the novel strain BRD125 in irradiated mice (gamma rays).

FIG. 11 illustrates the relative increase in the expression of hematopoietic cytokines in the spleen, by DeinoPol of the novel strain BRD125 in irradiated mice (gamma-rays).

FIGS. 12A to 12C illustrate the results of verifying the regeneration enhancing efficacy of leukocytes in peripheral blood (FIG. 12A), lymphocytes in peripheral blood (FIG. 12B) and immune cells (FIG. 12C) in the spleen, by DeinoPol of the novel strain BRD125 in irradiated mice (gamma rays).

### BEST MODE

Hereinafter, exemplary embodiments of the present disclosure will be described with reference to the accompanying drawings. However, embodiments of the present disclosure may be modified into various other forms, and the scope of the present disclosure is not limited to the embodiments described below.

The present inventors have identified a novel Deinococcus radiodurans strain having significantly improved exopolysaccharide production ability compared to the related art Deinococcus radiodurans, and confirmed that the exopolysaccharide produced from such a new strain has excellent antioxidant protection effect and UV protection effect even when comparing on the basis of the same amount as the Deinococcus radiodurans-derived exopolysaccharide of the related art.

Accordingly, according to an exemplary embodiment of the present disclosure, a novel Deinococcus radiodurans strain that may produce exopolysaccharides with improved productivity, and further produces exopolysaccharides having excellent antioxidant protection effects and UV protection effects is provided. In more detail, according to an exemplary embodiment of the present disclosure, there is provided a Deinococcus radiodurans BRD125 strain, characterized in being deposited with the accession number KCTC 13955BP.

In the present disclosure, “Deinococcus radiodurans-derived exopolysaccharide (EPS)” is referred to interchangeably with “DeinoPol”.

Further, according to an exemplary embodiment of the present disclosure, there is provided an exopolysaccharide derived from the Deinococcus radiodurans BRD125 strain deposited with the accession number KCTC 13955BP of the present disclosure.

An exopolysaccharide derived from the Deinococcus radiodurans BRD125 strain according to an exemplary embodiment includes arabinose, galactose, glucose and xylose.

In more detail, the exopolysaccharide includes 9% by weight of arabinose, 10% by weight of galactose, 15% by weight of glucose, 18% by weight of xylose, and 48% by weight of other unknown sugars, based on the weight of the total exopolysaccharide. In this case, the unknown other sugars include 32% by weight of other sugar 1 and 16% by weight of other sugar 2. Other sugars 1 and 2 above indicate unknown sugars detected as peaks other than the peaks of fucose, rhamnose, arabinose, galactose, glucose, xylose, and mannose, which are standard sugars in the sugar detection experiment by LC/MS. In more detail, in the present disclosure, the other sugar 1 is located between fucose and rhamnose based on the retention time in the sugar detection experiment by LC/MS, and the other sugar 2 is located between arabinose and galactose on the same basis. These other sugars may be analyzed to be a new type of unknown sugar by modifying the structure or residue of the sugar itself in the existing sugar.

Such a sugar combination of the exopolysaccharide of the present disclosure is a novel one that has not been previously known, and in more detail, there are no fucose and rhamnose which are present in the exopolysaccharide separated from Deinococcus radiodurans R1, the related art known strain, but there are two other types of sugars as described above.

Furthermore, according to an exemplary embodiment of the present disclosure, there is provided a cosmetic composition comprising the exopolysaccharide derived from the above-described Deinococcus radiodurans BRD125 strain.

The cosmetic composition may include the exopolysaccharide according to the exemplary embodiment of the present disclosure as an effective ingredient in a concentration of 0.8 μg/ml to 50 μg/ml, based on the volume of the total cosmetic composition, and in more detail, in a concentration of 3 μg/ml to 10 μg/ml.

If the exopolysaccharide is contained in an amount of less than 0.8 μg/ml based on the volume of the total cosmetic composition, the aging suppression and skin recovery effect is insufficient, and even in the case of 100 μg/ml or more, the use thereof may be possible since a change in toxicity of cells does not occur, but the exopolysaccharide may be included in a concentration of 50 μg/ml or less in terms of process economy because the increase in the effect due to the increase in content is not significant.

The cosmetic composition according to an exemplary embodiment of the present disclosure may be used for antioxidation, aging inhibition, radioprotection and/or skin recovery. In this case, the aging inhibition includes both the effect of preventing aging and reducing or delaying the progression of aging, and the skin recovery includes a series of processes in which the skin is protected from external stimuli such as UV light and damaged skin therefrom is restored.

For example, when the composition according to an exemplary embodiment of the present disclosure is a cosmetic composition, the cosmetic composition may be prepared in various forms, such as emulsions, lotions, creams (oil-in-water, water-in-oil, multi-phase), solutions, suspensions (anhydrous and aqueous), an anhydrous product (oil and glycol system), a gel, a mask, a pack, or a powder.

In addition to the active ingredient, the composition according to an embodiment of the present disclosure may contain additional ingredients including a carrier acceptable in cosmetic preparations and/or other additives generally formulated in cosmetics as needed.

In this case, “acceptable carrier in cosmetic formulations” refers to a compound or composition that is already known and used and may be included in a cosmetic formulation, or a compound or composition to be developed in the future, which does not have toxicity beyond the toxicity that may be adapted to the human body upon contact with the skin. The carrier may be included in the composition according to an exemplary embodiment in an amount of about 1% to about 99.99% by weight, in detail, about 50% to about 99% by weight of the total weight of the composition.

However, since the ratio varies depending on the above-described formulation of the cosmetic product and also varies according to a detailed application site or a preferred amount of application, the ratio should not be understood as limiting the scope of the present disclosure in any aspect.

On the other hand, as the additional ingredients, alcohol, oil, surfactant, fatty acid, silicone oil, wetting agent, moisturizing agent, viscosity modifier, emulsion, stabilizer, sunscreen agent, color developing agent, fragrance, antioxidant, plant extract, pH adjuster, pigment, or the like may be provided as an example. Compounds or compositions that may be used as alcohols, oils, surfactants, fatty acids, silicone oils, wetting agents, moisturizing agents, viscosity modifiers, emulsions, stabilizers, sunscreen agents, color developing agents, fragrances, or the like are already known in the art, and thus, an appropriate corresponding material or composition may be selected and used.

Further, according to an exemplary embodiment of the present disclosure, a pharmaceutical composition for preventing and inhibiting skin aging and a pharmaceutical composition for skin recovery and skin protection, including exopolysaccharide derived from the Deinococcus radiodurans BRD125 strain, are provided.

In more detail, based on the volume of the pharmaceutical composition, a pharmaceutical composition for preventing and inhibiting skin aging comprising 0.8 μg/ml to 50 μg/ml of Deinococcus radiodurans BRD125 strain-derived exopolysaccharide as an active ingredient, and a pharmaceutical composition for skin recovery, radioprotection and skin protection comprising 0.8 μg/ml to 50 μg/ml of exopolysaccharide derived from the Deinococcus radiodurans BRD125 strain as an active ingredient, are provided.

For example, the pharmaceutical composition according to an exemplary embodiment may be used as a pharmaceutical composition for the treatment of aging-related diseases, radioprotection and skin damage diseases, for example wrinkle, skin photoaging, pigmentation, freckle, melasma and skin damage by radiation such as UV.

According to a normal method based on the use thereof, the pharmaceutical composition according to an exemplary embodiment may be formulated and used in various forms, for example, oral formulations such as powders, granules, tablets, capsules, suspensions, emulsions, syrups, and aerosols, or the form of a sterile injection solution, and may be administered orally or through various routes including intravenous, intraperitoneal, subcutaneous, rectal, topical administration, and the like.

Examples of suitable carriers, excipients and diluents that may be included in the pharmaceutical compositions as described above may include lactose, dextrose, sucrose, sorbitol, mannitol, xylitol, erythritol, maltitol, starch, gum acacia, alginate, gelatin, calcium phosphate, calcium silicate, cellulose, methyl cellulose, amorphous cellulose, polyvinyl pyrrolidone, water, methylhydroxybenzoate, propylhydroxybenzoate, talc, magnesium stearate, mineral oil, and the like.

In addition, the therapeutic pharmaceutical composition may further include fillers, anti-aggregating agents, lubricants, wetting agents, flavoring agents, emulsifying agents, preservatives, and the like.

Solid preparations for oral administration include tablets, pills, powders, granules, capsules, and the like, and these solid preparations may be formulated by mixing at least one excipient, such as starch, calcium carbonate, sucrose, lactose, gelatin, or the like, in the pharmaceutical composition. Further, in addition to simple excipients, lubricants such as magnesium stearate and talc may also be used.

Liquid formulations for oral use include suspensions, liquid solutions, emulsions, syrups, and the like, and may include various excipients, such as wetting agents, sweeteners, fragrances, and preservatives, in addition to water and liquid paraffin, which are commonly used simple diluents.

Formulations for parenteral administration include sterile aqueous solutions, non-aqueous solvents, suspensions, emulsions, lyophilized formulations, and suppositories. As the non-aqueous solvent and suspending agent, propylene glycol, polyethylene glycol, a vegetable oil such as olive oil, and injectable ester such as ethyl oleate may be used. The base of the injection may include related art additives such as solubilizing agents, isotonic agents, suspending agents, emulsifying agents, stabilizing agents and preservatives.

The pharmaceutical composition according to an exemplary embodiment is administered or applied in a pharmaceutically effective amount to a subject in need of treatment for aging-related diseases and skin damage diseases.

The composition according to an exemplary embodiment may be administered as an individual therapeutic agent or administered in combination with other therapeutic agents, may be administered sequentially or simultaneously with a related art therapeutic agent, and may be administered single or multiple. It is important to administer an amount capable of obtaining a maximum effect in a minimum amount without side effects in consideration of all the above factors, which may be easily determined by a person skilled in the art. However, since the composition according to an exemplary embodiment may increase or decrease depending on the route of administration, the severity of the disease, sex, weight, age, or the ike, the dosage amount does not limit the scope of the present disclosure by any method.

Further, according to an exemplary embodiment of the present disclosure, there is provided an antioxidant food containing exopolysaccharide derived from a Deinococcus radiodurans BRD125 strain, as an active ingredient.

The antioxidant food according to an exemplary embodiment is a food composition that includes all health functional foods, health supplements, and the like, and the form of the food composition is not particularly limited, and any foods made in any form such as liquid, solid, or other fluid are intended to be included.

For example, when the food composition according to an exemplary embodiment is a food composition such as a functional beverage, the food composition may contain sweetening agents, flavoring agents, physiologically active ingredients, minerals, and the like, in addition to the active ingredients.

*59The sweetening agents may be used in an amount to give the food a suitable sweet taste, and may be natural or synthetic. In detail, a natural sweetener may be used, and examples of the natural sweetener may include sugar sweeteners such as corn syrup solids, honey, sucrose, fructose, lactose and maltose.

Flavoring agents may be used to enhance taste or aroma, and both natural flavors and synthetics may be used, and in detail, natural ones may be used. In the case of using natural ingredients, nutrient enhancement may be obtained in addition to flavor. A natural flavoring agent may be obtained from apples, lemons, tangerines, grapes, strawberries, peaches, or the like, or from green tea leaves, Solomon's seal, bamboo leaves, cinnamon, chrysanthemum leaves, jasmine, and the like. In addition, a natural flavoring agent obtained from ginseng (red ginseng), bamboo shoots, aloe vera, and ginkgo may be used. The natural flavoring agent may be a liquid concentrate or a solid extract. In some cases, synthetic flavoring agents may be used, and synthetic flavoring agents may include esters, alcohols, aldehydes, terpenes, and the like.

As the physiologically active substance, catechins, such as catechin, epicatechin, gallogatechin, and epigallocatechin, and vitamins such as retinol, ascorbic acid, tocopherol, calciferol, thiamine, riboflavin, and the like may be used. As minerals, calcium, magnesium, chromium, cobalt, copper, fluoride, germanium, iodine, iron, lithium, magnesium, manganese, molybdenum, phosphorus, potassium, selenium, silicon, sodium, sulfur, vanadium, zinc, and the like may be used.

In addition, the food composition according to an exemplary embodiment may contain preservatives, emulsifiers, acidulants, thickeners, and the like, if necessary, in addition to the sweetening agent. These preservatives, emulsifiers, and the like may be added and used in a very small amount as long as the application to which it is added may be obtained. When expressed numerically, the very small amount indicates a range of 0.0005% by weight to about 0.5% by weight based on the total weight of the food composition.

Preservatives that may be used include sodium calcium sorbate, sodium sorbate, potassium sorbate, calcium benzoate, sodium benzoate, potassium benzoate, ethylenediaminetetraacetic acid (EDTA), and the like. Examples of emulsifiers that may be used include gum acacia, carboxymethylcellulose, xanthan gum, and pectin. Examples of acidulants that may be used may include citric acid, malic acid, fumaric acid, adipic acid, phosphoric acid, gluconic acid, tartaric acid, ascorbic acid, acetic acid, and the like. These acidulants may be added so that the food composition has an appropriate acidity for suppressing the growth of microorganisms in addition to the use of enhancing taste. As a thickening agent that may be used, a suspending agent, a settling agent, a gel-forming agent, a swelling agent, and the like may be used.

On the other hand, according to another embodiment of the present disclosure, there is provided a method of extracting an exopolysaccharide derived from Deinococcus radiodurans BRD125. The extraction method includes obtaining a culture medium by culturing a Deinococcus radiodurans BRD125 strain; precipitating the exopolysaccharide in the culture medium using 50% or more of ethanol; an impurity removal operation of removing protein, lipid and a nucleic acid as precipitates from a precipitate of the precipitating; and purifying a supernatant obtained in the impurity removal operation.

In the operation of obtaining a culture medium by culturing Deinococcus radiodurans, the culture temperature may be a temperature of 28° C. to 38° C., in detail, a temperature of 30 to 35° C., in more detail, about 30° C.

The operation of obtaining the culture medium may be performed by, for example, inoculating Deinococcus radiodurans in 2L of Tryptone-Glucose-Yeast extract (TGY) and then, shaking incubating for 48 hours at a temperature of about 30° C., and then, irradiating the suspension with gamma rays for 2 hours at 17 kGy to separate and sterilize the EPS of the cell wall of Deinococcus radiodurans in the cultured medium, and then, removing the killed Deinococcus radiodurans by centrifugation.

Further, subsequent to the operation of obtaining the culture medium, an operation of concentrating the obtained culture medium 50 times to 200 times, using an ultrafiltration system, in detail, 80 times to 130 times concentration, may further be included in the extraction method. In the case of performing the concentration as described above, a relatively large amount of exopolysaccharide may be extracted more efficiently.

Subsequently, the operation of precipitating the exopolysaccharide in the culture medium using an aqueous ethanol solution of 50% or more of ethanol concentration may be performed. For example, the culture medium is mixed with ethanol having a concentration of 50% or more, in detail, with an aqueous ethanol solution having a concentration of 50% or more and less than 80%, and then, after about 1 hour to 24 hours, in detail, 10 to 14 hours, at 0 to 4° C. elapses, nucleic acid and high molecular weight substances are precipitated and may be removed through centrifugation.

Further, in the supernatant obtained as above, the ethanol concentration in the aqueous ethanol solution may be increased to 80% or more, to then be precipitated at 0 to 4° C. for about 1 hour to 24 hours, in detail, for 10 hours to 14 hours, such that exopolysaccharide in the supernatant may be additionally precipitated. If the ethanol concentration of the culture medium is less than 80%, there is a problem in which low molecular weight EPS does not precipitate.

The exopolysaccharide precipitated by the processes above may be mixed with water to prepare an aqueous exopolysaccharide solution. The aqueous solution has a form in which exopolysaccharide is dissolved in water. Insoluble substances are not water-soluble exopolysaccharides, and thus, may be removed through solid-liquid separation, for example, using a filter. In this case, the filters that may be used are not particularly limited as long as they may remove insoluble substances, and for example, insoluble substances may be removed using a 0.22 μm filter.

The precipitate includes not only exopolysaccharide, but also other proteins or lipids, and the impurity removal operation performed to remove these precipitates may be performed by adding an aqueous mixed alcohol solution containing chloroform and butanol in a weight ratio of 4:1 to the precipitate to obtain exopolysaccharide as a supernatant. In this case, the aqueous mixed alcohol solution may be a mixture in which mixed alcohol and water are mixed in a weight ratio of 3:7 to 7:3, in detail, in a weight ratio of 5:5.

On the other hand, the operation of purifying the supernatant may be performed by dialysis. For example, the separated supernatant may be dialyzed in tertiary sterile distilled water using a 10 kDa dialysis tube.

Alternatively, the purification operation may be performed by gel filtration. The gel filtration method applied to the purification operation is not particularly limited. For example, after stabilizing a Sephacryl S-300HR column with secondary distilled water in a refrigerator at a temperature of 4° C., 5 ml of precipitated exopolysaccharide is injected into the column, and after the injection, secondary distilled water is continuously injected at a rate of 5 ml/min to obtain an eluate separated through the column. At this time, the eluent may be confirmed as an eluent from which exopolysaccharide is produced by measuring the concentration of sugar using, for example, an anthrone reaction sugar analysis method or the like.

The method of extracting an exopolysaccharide according to an exemplary embodiment of the present disclosure may simplify the purification process and increase purification efficiency.

Furthermore, an operation of drying the purified exopolysaccharide may be further included. In this case, the drying method is not particularly limited, and may be performed, for example, by freeze drying.

As described above, according to an exemplary embodiment of the present disclosure, a method for extracting exopolysaccharide from the Deinococcus radiodurans BRD125 strain is provided, and by the process described as above, exopolysaccharide (EPS) having a composition of a new polysaccharide derived from a novel Deinococcus radiodurans BRD125 strain may be obtained with a high yield, and in addition, a composition having a significantly improved skin aging prevention and skin recovery action may be implemented.

Hereinafter, the present disclosure will be described in more detail through specific examples. The following examples are only examples to aid understanding of the present disclosure, and the scope of the present disclosure is not limited thereto.

### MODE FOR INVENTION

### Example

1. Isolation of Radiation-Resistant Microorganisms from Baengnokdam in Hallasan Mountain

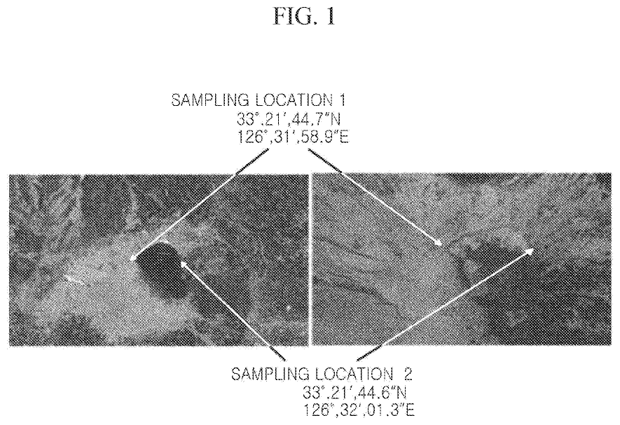

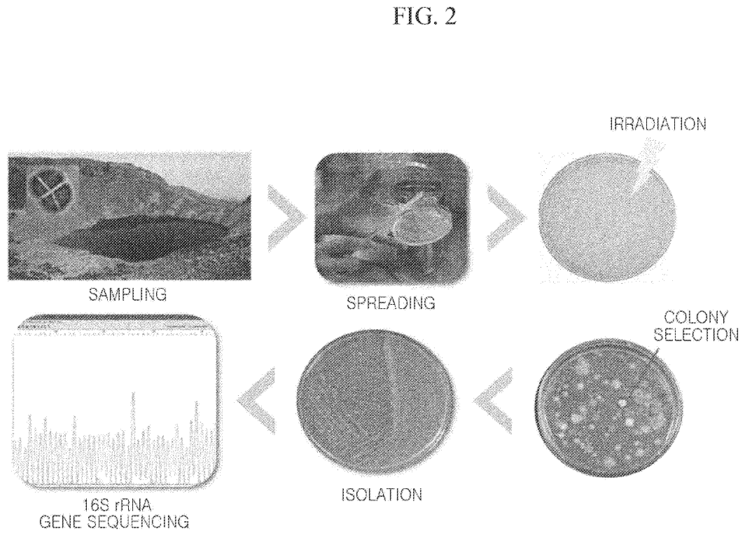

About 30 g of soil was collected from two places in Baengnokdam (see FIG. 1, Table 1) in Hallasan Mountain, and gamma rays of 5kGy was irradiated using a high-level radiation irradiation facility in the Advanced Radiation Research Institute of Korea Atomic Energy Research Institute. 30 ml of R2A liquid medium was added to the soil irradiated with gamma rays, to then be incubated (enrichment) for 2 hours at 30° C., and then, respective about 0.5 ml was smeared in R2A solid medium to then be incubated in an incubator at 30° C. for 3 days. Colonies appearing on the R2A solid medium were selected and incubated for two days in the R2A liquid medium. A portion of the cultured liquid medium produced a storage strain, and the remainder was spread on R2A solid medium again, cultured in an incubator at 30° C. for two days, and colonies were confirmed on R2A solid medium (see FIG. 2).

Through 16s rRNA analysis, radiation-resistant microorganisms of 58 species from soil, 49 species from clay and 3 species from freshwater were isolated and identified. Finally, 110 radiation-resistant microorganisms were isolated.

TABLE 1
Soil collection coordinates

| | Coordinates |
|---|---|
| Sampling location 1 | 33°, 21′, 44.7″N 126°, 31′ 58.9″E |
| Sampling location 2 | 33°, 21′, 44.6″N 126°, 32′ 01.3″E |

2. Identification of Deinococcus Radiodurans Through 16s rRNA Analysis

In order to identify 110 radiation-resistant microorganisms isolated by the process of 1 above, genomic DNA was isolated, and 16S rRNA gene was analyzed (see Table 2). 16S rRNA gene sequence analysis was performed using 27F(5′-AGAGTTTGATCMTGGCTCAG-3) and 1492R primers (5′-TACGGYTACCTTGTTACGACTT-3′) to analyze the nucleotide sequence of the PCR product amplified by about 1,350 bp or more. To confirm the similarity between the base sequences, the isolated radiation resistant microorganisms were identified using NCBI's BLAST database. As a result, out of 110 strains, 30 species having 99% homology to Deinococcus radiodurans (D. radiodurans) were identified.

As a result, out of 110 strains, 30 species of D. radiodurans were identified, and three thereof were confirmed to be highly likely to be new species.

TABLE 2
16s mRNA analysis result and identified microorganism name

| | Origin(Hallasan | | | | |
|---|---|---|---|---|---|
| RRMB Code | Baeknokdam) | Strain name | Identities(%) | Remark* | |
| RRMB-00065 | Gwaneumsa | Spirosoma sp. | 92 | | |
| | Temple Entrance | | | | |
| RRMB-00066 | Gwaneumsa | Spirosoma rigui | 93 | | |
| | Temple Entrance | | | | |
| RRMB-00067 | Gwaneumsa | Spirosoma oryzae | 98 | | |
| | Temple Entrance | | | | |
| RRMB-00075 | Fresh Water | Staphylococcus epidermidis | 99 | 3 | species |
| RRMB-00076 | Fresh Water | Microbacterium oxydans | 99 | | |
| RRMB-00077 | Fresh Water | Promicromonospora iranensis | 99 | | |
| RRMB-00078 | Clay | Hymenobacter soli | 96 | 5 | species |
| RRMB-00080 | Clay | Staphylococcus aureus | 99 | | |
| RRMB-00081 | Clay | Paenibacillus typhae | 99 | | |
| RRMB-00082 | Clay | Hymenobacter ginsengisoli | 96 | 18 | species |
| RRMB-00084 | Clay | Deinococcus radiodurans | 99 | 30 | species |
| RRMB-00085 | Clay | Geodermatophilus brasiliensis | 97 | | |
| RRMB-00086 | Clay | Aurantimonas sp. | 97 | | |
| RRMB-00087 | Clay | Spingomonas dokdonensis | 97 | | |
| RRMB-00090 | Clay | Brevundimonas subvibrioides | 98 | 4 | species |
| RRMB-00097 | Clay | Sphingomonasdaceae bacterium | 99 | 3 | species |
| RRMB-00101 | Clay | Bosea lupini | 99 | | |
| RRMB-00125 | Clay | Leuconostoc gelidum | 100 | | |
| RRMB-00127 | Soil | Hymenobacter metalli | 95 | 8 | species |
| RRMB-00128 | Soil | Hymenobacter sp. | 95 | 3 | species |
| RRMB-00133 | Soil | Deinococcus sp. | 96 | 4 | species |
| RRMB-00140 | Soil | Methylobacterium extorquens | 99 | 4 | species |
| RRMB-00141 | Soil | Deinococcus altitudinis | 96 | 2 | species |
| RRMB-00147 | Soil | Novosphingobium stygium | 98 | 2 | species |
| RRMB-00151 | Soil | Aquabacterium parvum | 98 | | |
| RRMB-00154 | Soil | Paenibacillus terrae | 99 | | |
| RRMB-00155 | Soil | Aureimonas altamirensis | 99 | | |
| RRMB-00165 | Soil | Novosphingo stygium | 97 | | |
| RRMB-00168 | Soil | Novosphingobium fuchskuhlense | 99 | | |
| RRMB-00170 | Soil | Methylobacterium marchantiae | 99 | | |
| RRMB-00171 | Soil | Salmonella enterica | 99 | | |
| RRMB-00172 | Soil | Staphylococcus saccharolyticus | 99 | | |
| RRMB-00173 | Soil | Methylobacterium phylloshaerae | 99 | 3 | species |
| RRMB-00174 | Soil | Gordonia broncgialis | 100 | | |

3. Confirmation of Exopolysaccharide Production of D. radiodurans (Comparison of DeinoPol Production)

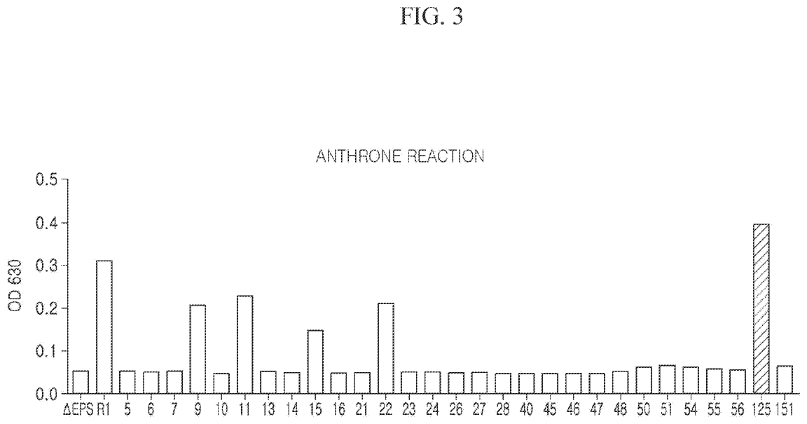

The Deinococcus radiodurans strains and the Deinococcus radiodurans R1 (by US ATCC) strain isolated by the process as in 1 above and identified by the process as described in 2 above were inoculated into TY liquid medium, respectively, were cultured under the conditions of 30° C. and 200 rpm, and were centrifuged to separate the culture medium obtained by culturing the Deinococcus radiodurans strains. Anthrone reaction was performed to confirm the Deinococcus radiodurans-derived exopolysaccharide (EPS) (referred to interchangeably with “DeinoPol”) in the isolated culture medium. Each bar graph of FIG. 3 represents the OD630 value of DeinoPol derived from a strain having a name including the number at a lower end of the corresponding bar graph, and ΔEPS represents a mutant strain that cannot produce DeinoPol.

As a result, it was confirmed that a lot of EPS was produced in the Deinococcus radiodurans R1, BR9, BR11, BR15, BR22, and BRD125 strains, and a relatively largest amount of EPS was produced in the Deinococcus radiodurans BRD125 strain. The results are illustrated in a graph in FIG. 3, and in the graph of FIG. 3, “BR” is omitted in front of the number in relation to the name of each strain, and in the case of 125, “BRD” is omitted.

The thus-obtained Deinococcus radiodurans BRD125 strain was deposited with the Korea Research Institute of Bioscience and Biotechnology, Biological Resource Center, under the accession number KCTC 13955BP on Sep. 17, 2019.

4. Isolation and Purification of DeinoPol from D. Radiodurans BRD125

In order to isolate DeinoPol of Deinococcus radiodurans BRD125, which produced the most exopolysaccharides in 3 above, incubation in TGY liquid medium at 30° C. and 200 rpm for 2 days, and centrifugation were performed to obtain a culture medium. The obtained culture medium was concentrated 10 to 20 times using a rotary evaporator at 65° C. The concentrated culture medium was 50% ethyl alcohol aqueous solution, and DeinoPol was precipitated at 4° C. for 12 hours to remove nucleic acid and some protein precipitates through centrifugation to obtain a supernatant.

The concentration of ethyl alcohol in the supernatant was raised from 50% to 80% and was precipitated at 4° C. for 12 hours, and the precipitated DeinoPol precipitate was separated through centrifugation and then dissolved in tertiary distilled water.

The precipitate contains not only DeinoPol, but also other proteins or lipids, and for the removal thereof, a 4:1=chloroform:butanol mixed alcohol solution was mixed with DeinoPol dissolved in tertiary sterilized distilled water at 1:1, and then, a DeinoPol-contained supernatant was separated through centrifugation.

The separated supernatant was dialyzed in tertiary sterilized distilled water using a 10 kDa dialysis tube, and then freeze-dried to obtain a final DeinoPol.

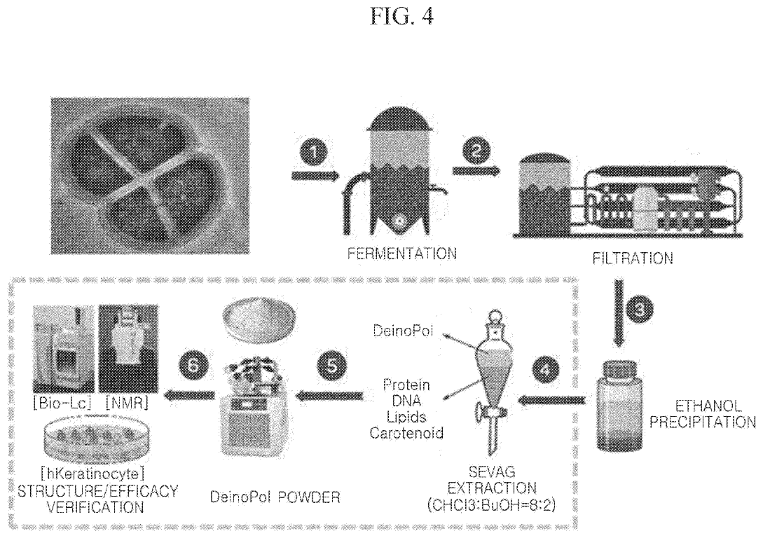

The above process is schematically illustrated in FIG. 4, and the resulting DeinoPol powder was well purified with a purity of 94.7%. (see Table 3)

TABLE 3
Purity of Purified DeinoPol

| | EPS | DNA | Protein | EPS purity |
|---|---|---|---|---|
| D. Radiodurans | 94.75 mg/L | 0.125 mg/L | 1.967 mg/L | 94.7% |
| BRD125 | | | | |

5. Confirmation of Sugar Composition of Small Polysaccharide Derived from Deinococcus Radiodurans BRD125

The sugar composition of the EPS derived from Deinococcus radiodurans BRD125, extracted in the process of 4 above, was analyzed using BIO-LC, and the results are illustrated in Table 4 below. Other sugars illustrated in Table 4 below are unknown sugars detected, other than the peaks of fucose, rhamnose, arabinose, galactose, glucose, xylose, and mannose, which were standard sugars during the sugar detection experiment by LC/MS. In more detail, in the present disclosure, other sugar (1) is located between fucose and rhamnose based on the retention time during the sugar detection experiment by LC/MS, and other sugar (2) is located between arabinose and galactose on the same basis. These other sugars may be analyzed to be a new type of unknown sugar by modifying the structure or residue of the sugar itself in the existing sugar.

As can be seen in Table 4 below, the sugar composition of the EPS derived from Deinococcus radiodurans BRD125 is as follows:

TABLE 4

| Type of sugar | Content(Wt %) |
|---|---|
| Arabinose | 9 |
| Galactose | 10 |
| Glucose | 15 |
| Xylose | 18 |
| Unknown other sugar 1 | 32 |
| Unknown other Sugar 2 | 16 |
| Sum | 100 |

As a result of the analysis, as illustrated in Table 4, it was confirmed that the sugar composition of the EPS derived from Deinococcus radiodurans BRD125 was a previously unknown sugar composition.

6. Comparison of Antioxidant Efficacy of DeinoPol of Deinococcus Radiodurans BRD125

(1) Comparison of Antioxidant Ability Through DPPH Experiment

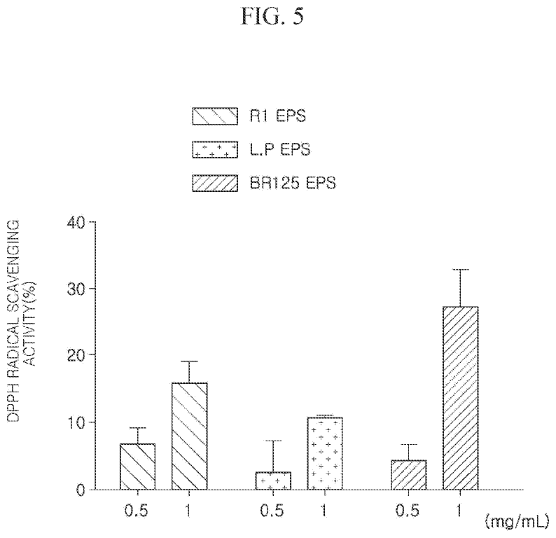

In order to compare the antioxidant capacity of exopolysaccharide (L.P EPS) of Lactococcus plantarum (L.P), which is a control group known to have antioxidant capacity, and purified DeinoPol BRD125 (BRD125 EPS) and DeinoPol R1 (R1 EPS), the DPPH experiment was carried out, and the results are illustrated in FIG. 5.

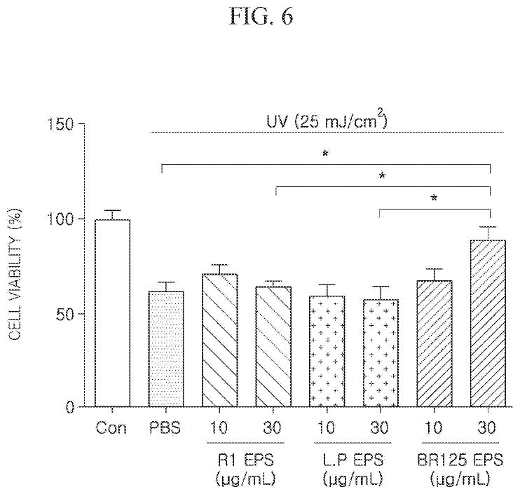

In addition, to confirm the protective effect of each DeinoPol against UV, HEKa cells were seeded and stabilized for 18 hours, treated with DeinoPol, and cultured for 12 hours. At 30 minutes before UV irradiation, HEKa cells were replaced with the fresh medium without FBS and was irradiated with UV. After 4 hours after irradiation, it was changed to the fresh medium containing PBS, and after 3 days, cell viability was calculated using CCK-8, and the results are illustrated in FIG. 6.

As a result, as can be seen in FIGS. 5 and 6, respectively, as a result of comparing the efficacy for the same amount of exopolysaccharide, it was confirmed that DeinoPol BRD125 (BRD125 EPS) had a significantly high antioxidant effect, and had excellent protective effect against UV.

(2) Checking Effectiveness of Removing Reactive Oxygen Species (ROS)

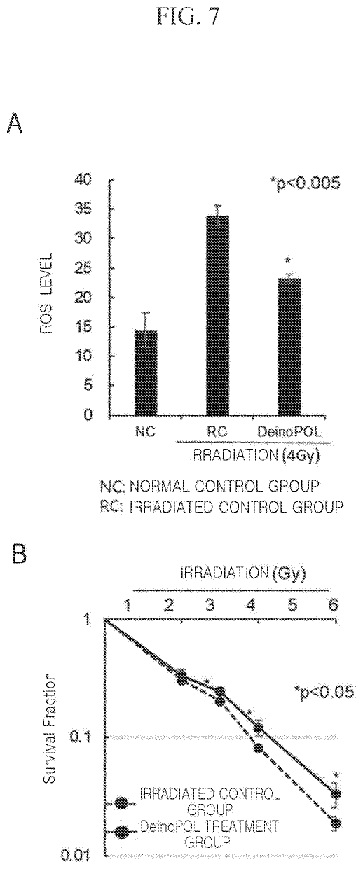

In order to confirm the effectiveness of radiation-induced reactive oxygen species (ROS) removal by DeinoPol of the novel strain BRD125, CHO cells were seeded and stabilized for 18 hours and then were treated with DeinoPol at a concentration of 50 μg/ml. At 4 hours after DeinoPol treatment, DCF-DA, a reagent that reacts with reactive oxygen species, was treated at a concentration of 20 μM for 1 hour, and then, the cells were isolated and immediately irradiated with X-rays. The concentration of reactive oxygen species in the cells was analyzed using a flow cytometry. As a result, it was confirmed that relatively very high reactive oxygen species was measured in the irradiated control group, while the reactive oxygen species was significantly reduced by the treatment of DeinoPol (see FIG. 7A).

In order to confirm the radioprotective efficacy by DeinoPol of the new strain BRD125, 200-500 CHO cells were seeded per well of a 6-well plate and stabilized for 3 hours, and then treated with DeinoPol at a concentration of 50 μg/ml. At 2 hours after DeinoPol treatment, 2, 3, 4 and 6Gy were irradiated under the conditions of X-ray 160 kV and 1 mA, respectively. On the 7th day after X-ray irradiation, the culture medium was discarded, the cells were fixed with methanol, and then, the colonies were stained using 0.5% crystal violet and counted. At this time, the CHO cell viability calculation method is as follows:

Survival Fraction=PE of the test group/PE of the normal control group

*PE(Plating Efficiency)=counted colonies/starting cellsX100

As a result, it was confirmed that the survival fraction in CHO cells after X-ray irradiation was statistically significantly increased by DeinoPol treatment, compared to the irradiated control group (FIG. 7B).

7. Confirmation of Radioprotective Efficacy of DeinoPol of Deinococcus Radiodurans BRD125

(1) Confirmation of Efficacy of Inhibiting Apoptosisof Hematopoietic Cells and Immune Cells by Radiation In Vitro

*119In order to confirm the radioprotective efficacy by DeinoPol of the novel strain BRD125 in the radiation-sensitive hematopoietic cells and immune cells, the bone marrow (hematopoietic cells) and spleen (immune cells) of mice were collected, and the cells were respectively isolated therefrom. Each cell was seeded in a 12-well plate and treated with DeinoPol at a concentration of 50 μg/ml for 3 hours, followed by X-ray irradiation at a dose of 3 Gy under 160 kV and 1 mA conditions for bone marrow cells and 1 Gy for immune cells in the spleen. At 24 hours after X-ray irradiation, all cells were collected, fixed in 70% ethanol and stained with Propidium iodide (PI) reagent, and the cell cycle and the percentage of dead cells were analyzed using a flow cytometry. As a result, the cell death rate of both bone marrow cells and splenocytes significantly increased by irradiation, whereas cells in the G1 phase decreased. It was confirmed that, in both bone marrow cells and splenocytes, the group treated with DeinoPol had a statistically significant decrease cell death rate compared to the irradiated control group, and the G1 phase had an increase therein, compared to the irradiated control group (FIG. 8A, 8B). It was confirmed through this experiment that DeinoPol treatment increases the expression of BCL-2, a cell death inhibitory factor, reduced by irradiation in bone marrow cells and splenocytes (FIG. 8C).

(2) Confirmation of Efficacy of Inhibiting Death of Hematopoietic Cells and Immune Cells by Radiation In Vivo

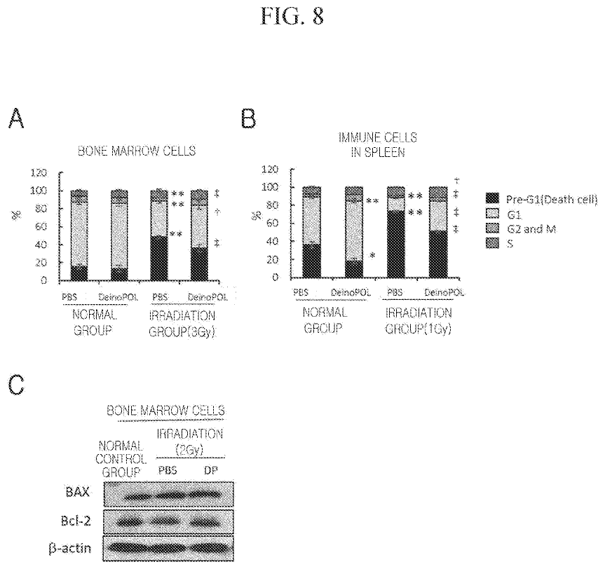

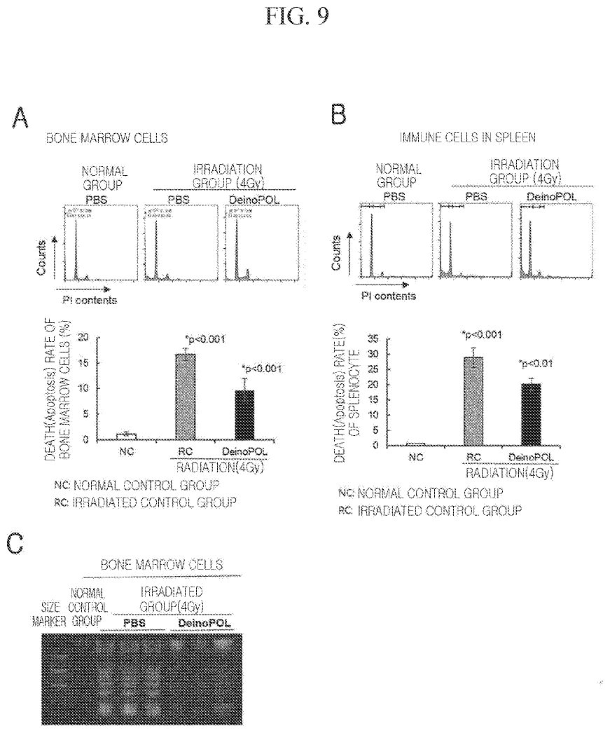

In vivo, in order to confirm the radioprotective efficacy of DeinPol of the new strain BRD125 to the hematopoietic system and immune cells, DeinoPol was intraperitoneally injected at 50 μg/kgBW 48 and 24 hours before radiation (gamma rays) irradiation and within 30 minutes immediately after irradiation to mice, and the radiation exposure was performed as a single whole body irradiation at a dose of 4 Gy. At four hours after irradiation, all mice were sacrificed and then bone marrow cells and splenocytes were collected, fixed with 70% ethanol, stained with PI (Propidium iodide) reagent, and the percentage of apoptotic cells was analyzed using a flow cytometry. In addition, fragmented DNA from the bone marrow cells was collected and were then electrophoresed in a 2% agarose gel to confirm the degree of DNA fragmentation of the apoptotic hematopoietic cells by radiation. As a result, it was confirmed that the cell death rate was significantly increased by irradiation in both bone marrow cells and splenocytes, but the radiation-induced death rate was statistically significantly reduced by administration of DeinoPol (see FIG. 9A and FIG. 9B). In addition, it was confirmed that the DNA fragmentation of bone marrow cells was significantly higher than that of the normal control group by irradiation, but the DNA fragmentation phenomenon was decreased in the bone marrow cells of mice administered with DeinoPol (FIG. 9C). Therefore, it was found that DeinoPol has excellent protective effect on hematopoietic stem cells and immune cells in irradiated mice.

(3) Confirmation of Increased Endogenous Splenic Colony Formation by DeinoPol in Irradiated Mice

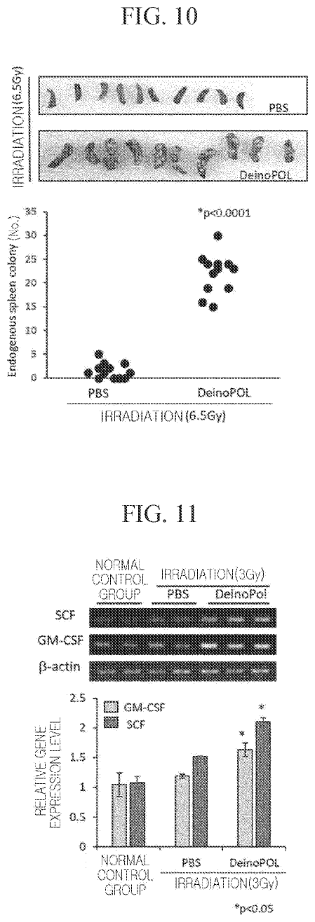

Radiation-sensitive hematopoietic stem cells are rapidly lost after irradiation, and then, an emergency hematopoietic action occurs in the spleen to supplement the number of cells in the blood, and hematopoietic cell colonies may be observed on the spleen surface. To confirm the increase in endogenous splenic colony formation by DeinoPol of the novel strain BRD125 in irradiated mice, DeinoPol was intraperitoneally injected at 50 μg/kgBW, 48 and 24 hours before radiation (gamma rays) irradiation and within 30 minutes and after 24 and 48 hours immediately after irradiation, and the irradiation was carried out with a single whole body irradiation at a dose of 6.5 Gy. On the 9th day after irradiation, all mice were sacrificed, the spleens were collected, fixed and stained with Bouin's solution. The number of colonies formed on the spleen surface was counted. As a result, it was confirmed that the number of hematopoietic cell colonies on the spleen of the irradiated control group was an average of 1.5, whereas an average number is 22.8 in irradiated mice administered with DeinoPol (see FIG. 10). Therefore, it was found that DeinoPol has an excellent effect of promoting the protection and regeneration of hematopoietic stem cells in irradiated mice.

(4) Confirmation of Effects on Expression of Hematopoietic Factors by Radiation In Vivo

To confirm the increase in the expression of the hematopoietic factors GM-CSF and SCF by DeinoPol in irradiated mice, DeinoPol was intraperitoneally injected at 50 μg/kgBW, 48 and 24 hours before radiation (gamma rays) exposure and within 30 minutes and 24 and 48 hours after irradiation, and the irradiation was performed by single whole body irradiation at a dose of 3Gy. On the 7th day after irradiation, all mice were sacrificed, cells were collected from the spleen, and RNA was extracted. The extracted RNA was synthesized as cDNA through the RT process, and the expression levels of GM-CSF and SCF were compared by performing a PCR process. As a result, it was confirmed that the expression of GM-CSF and SCF increased by 1.5 times and 2 times in irradiated mice injected with DeinoPol, compared to the irradiated control group, respectively (FIG. 11).

(5) Confirmation of Effects on Recovery of Number of Peripheral Blood Cells and Spleen Immune Cells after Irradiation In Vivo

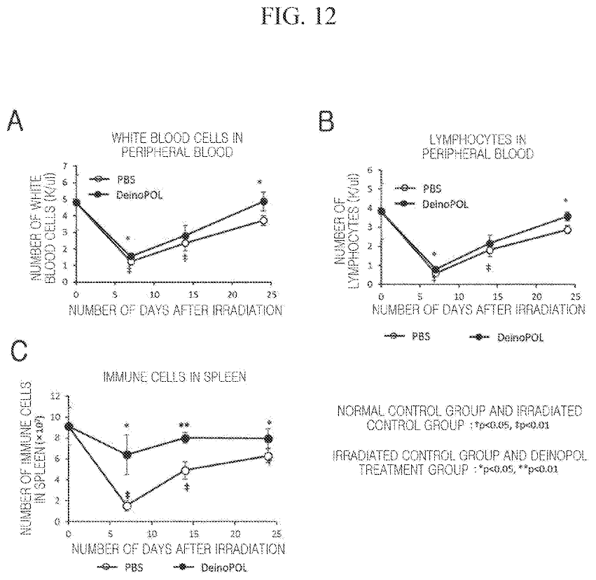

After irradiation, the number of white blood cells and lymphocytes in the peripheral blood, and the number of immune cells in the spleen, rapidly decrease. In this case, recovery of the number of immune cells by regeneration of immune cells plays an important role in recovery after irradiation. Therefore, the effect of DeinoPol on the recovery of the number of peripheral blood and spleen immune cells was observed after irradiation at a dose of 3Gy, a sublethal dose. To confirm the increase in recovery of (a) the number of white blood cells in the peripheral blood, (b) the number of lymphocytes in the peripheral blood, and (c) the number of immune cells in the spleen by DeinoPol of the novel strain BRD125 in irradiated mice, DeinoPol was intraperitoneally injected at 50 μg/kgBW, 48 and 24 hours before irradiation and within 30 minutes and 24 and 48 hours after radiation (gamma rays) exposure at a dose of 3 Gy. Blood was collected through the orbital vein of mice on 7-th, 14-th, and 21-th days after irradiation, and the number of white blood cells and lymphocytes in the peripheral blood was measured using an automatic hemocytometer, and immune cells in the spleen were collected and counted. As a result, it was observed that in the irradiated control group, the white blood cells and lymphocytes in the peripheral blood on the 7th day after irradiation decreased sharply to the level of about 20% of the normal control group, and then gradually recovered and returned to the pre-irradiation level 3 weeks after irradiation. On the other hand, in the irradiated group administered with DeinoPol, the number of white blood cells and lymphocytes was statistically significantly higher than that of the irradiated control group, and regeneration was also increased (FIGS. 12A and 12B). In addition, as a result of counting the number of immune cells in the spleen, in the irradiated control group, the number of immune cells rapidly decreased on the 7th day after irradiation and gradually recovered, whereas in the irradiated mice administered with DeinoPol, the number of immune cells decreased slightly on the 7th day after irradiation, but there was almost no difference from the normal control group at the 14th day (FIG. 12C). From these results, it was confirmed that DeinoPol has an effect of promoting the recovery of the number of immune cells after irradiation.

Although the embodiments of the present disclosure have been described in detail above, the scope of the present disclosure is not limited thereto, and various modifications and variations are possible without departing from the technical spirit of the present disclosure described in the claims, which will be obvious to those of ordinary skill in the art.

1. (canceled)

2. An exopolysaccharide comprising arabinose, galactose, glucose, xylose and fructose.

3. The exopolysaccharide of claim 2, wherein the exopolysaccharide comprises 9% by weight of arabinose, 10% by weight of galactose, 15% by weight of glucose, 18% by weight of xylose, and 48% by weight of other unknown sugars, based on a weight of a total exopolysaccharide.

4. The exopolysaccharide of claim 2, wherein the exopolysaccharide is derived from a Deinococcus radiodurans BRD125 strain deposited with accession number KCTC 13955BP.

5. A composition comprising the exopolysaccharide of claim 2.

6. The composition of claim 5, wherein the composition is a cosmetic composition comprises the exopolysaccharide in a concentration of 0.8 μg/ml to 50 μg/ml based on a volume of a total cosmetic composition.

7. The composition of claim 6, wherein the cosmetic composition is for aging inhibition and skin recovery.

8. The composition of claim 5, wherein the composition is a pharmaceutical composition for inhibiting skin aging, comprising the exopolysaccharide as an active ingredient.

9. The composition of claim 5, wherein the composition is a pharmaceutical composition for skin recovery comprising the exopolysaccharide as an active ingredient.

10. The composition of claim 5, wherein the composition is an antioxidant food composition comprising the exopolysaccharide as an active ingredient.

11. A method of extracting an exopolysaccharide derived from Deinococcus radiodurans, the method comprising:
obtaining a culture medium by culturing a Deinococcus radiodurans BRD125 strain;
precipitating the exopolysaccharide in the culture medium, using an ethanol aqueous solution having an ethanol concentration of 50% or more;
Removing impurity from a precipitate of the precipitating by removing protein, lipid, and a nucleic acid as precipitates; and
purifying a supernatant obtained from the removing impurity.

12. The method of claim 11, wherein the removing impurity is performed by adding a mixed alcohol aqueous solution containing chloroform and butanol in a weight ratio of 4:1 to the precipitate to obtain the exopolysaccharide as a supernatant.

13. The method of claim 12, wherein the purifying the supernatant is performed by dialysis.

14. The method of claim 11, further comprising drying a purified exopolysaccharide.

15. The method of claim 11, wherein the Deinococcus radiodurans strain is a Deinococcus radiodurans BRD125 strain.
